(19) United States
(12) Patent Application Publication
(10) Pub. No.: US 2025/0143773 A1
(43) Pub. Date: May 8, 2025

(54) ELECTROSURGICAL INSTRUMENT WITH COMMUNICATION INTERFACE AND ELECTROSURGICAL GENERATOR

(71) Applicant: OLYMPUS WINTER & IBE GMBH, Hamburg (DE)

(72) Inventor: Jens KRÜGER, Zeuthen (DE)

(73) Assignee: OLYMPUS WINTER & IBE GMBH, Hamburg (DE)

(21) Appl. No.: 18/941,620

(22) Filed: Nov. 8, 2024

(30) Foreign Application Priority Data
Nov. 8, 2023 (DE) 102023131006.2

(51) Int. Cl. A61B 18/12; A61B 18/00

(52) U.S. Cl. CPC A61B 18/1206; A61B 2018/00077; A61B 2018/00178; A61B 2018/00916

(57) ABSTRACT

Electrosurgical instrument having a HF energy cable and a memory including status information wireless communication means configured for communicating data signals between said memory and the electrosurgical generator. The electrosurgical instrument further includes a signal processing device configured for gathering and communicating gathered data of said electrosurgical instrument. The signal processing device is connected to the wireless communication means and includes at least one sensor and/or actuator and an energy harvesting circuit. Said energy harvesting circuit is configured for harvesting energy wirelessly from the HF energy supplied by the cable. Thereby energy for operating the signal processing device and/or the at least one sensor and/or the at least one actuator is provided wirelessly.

The invention relates to an electrosurgical instrument configured for being supplied with high-frequency (HF) energy via a HF energy cable from an electrosurgical generator. The electrosurgical instrument further comprises a memory in which status information is stored and wireless communication means configured for communicating data signals between said memory and a corresponding wireless communication front end of the electrosurgical generator.

In electrosurgery, an electrosurgical instrument such as an electroscalpel is used to apply high-frequency alternating current to tissue in the human body. Usually high-frequency in the radiofrequency range of about 200 kHz to up to 4,000 KHz is used. This results in local heating of the tissue. Thereby, the tissue is cut or severed by heating, and the tissue is removed by thermal resection. A major advantage of electrosurgery is that bleeding can be stopped at the same time as the cutting is made by closing the affected vessels, and electrosurgical instruments can be used for other applications, such as coagulation.

Electrosurgery is used for a variety of tasks in different fields of surgery, and various kinds and types of electrosurgical instruments are connected to the electrosurgical generator. In consideration of the sensitivity of the tissue and its surroundings which are to be treated by electrosurgery, the electrosurgical instrument to be used must be fit for the surgery to be accomplished which requires close monitoring and controlling in order to ensure a safe and efficient surgical procedure. Specifically, the electrosurgical instrument must be the right one and it must be in a condition which ensures that said instrument is still fit for the task at hand.

In order to accomplish this, it is known that the electrosurgical instrument is provided with a wireless communication means, in particular a RFID transponder, which interacts with the corresponding frontend for wireless communication of the electrosurgical generator, e.g. an electrosurgical generator of the ESG-200 family as produced by Olympus Winter & Ibe GmbH (Hamburg, Germany). By this wireless communication means, the electrosurgical generator can read and write to a memory of the electrosurgical instrument's transponder. Thereby, data concerning the type of the electrosurgical instrument as well as usage data of the instrument can be communicated. The energy necessary for operating the memory is conveyed by the RFID transponder, thereby allowing the electrosurgical instrument to not depend on an internal battery. However, due to this design the energy available is rather limited and functionality is essentially restricted to transferring a small quantity of stored data that is static or nearly static. This configuration is not enabled to transfer dynamically changing data nor real-time data.

It is thus an object of the invention to provide improved electrosurgical instrument being enabled for communicating more and fast-changing data.

The solution according to the invention resides in the features of the independent claim. Advantageous embodiments are the subject matter of the dependent claims.

An electrosurgical instrument having a HF energy cable configured for being supplied with HF energy from an electrosurgical generator, the electrosurgical instrument comprising a memory comprising status information of the said electrosurgical instrument and wireless communication means configured for communicating data signals between said memory and a corresponding wireless communication frontend of the electrosurgical generator, it is provided according to the invention that the electrosurgical instrument further comprises a signal processing device configured for gathering and communicating gathered data of said electrosurgical instrument, the signal processing device being communicatively connected to the wireless communication means and comprising at least one sensor and/or actuator at the electrosurgical instrument, and an energy harvesting circuit configured for wirelessly harvesting energy from the HF energy supplied by the cable, said energy harvesting circuit being a power supply for the signal processing device and/or the at least one sensor and/or the at least one actuator.

The invention has realized that the key to an improved data transmission, i.e. more and dynamically changing data, is providing sufficient energy for onboard data processing at the electrosurgical instrument, and thereby any need for batteries or separate power cabling can be avoided.

A core aspect of the invention is to furnish an energy harvesting circuit that is enabled to draw energy from the large amount of HF energy supplied to the electrosurgical instrument by the electrosurgical generator. This HF energy passes through the cable connecting said electrosurgical instrument with the electrosurgical generator and through the electrosurgical instrument every time the electrosurgical instrument is to be activated. The electrosurgical instrument applies the supplied HF energy for treating of tissue, particularly by local heating of the tissue. Thereby, the tissue is typically cut or severed by heating (thermal resection), and bleeding is stopped by a thermal sealing effect, as already explained above in the introductory portion to the prior art. For transferring the HF energy to the tissue the electrosurgical instrument may be provided with an HF applicator, e.g. a blade or a pincer. Due to rather high voltage used for supplying the HF energy (up into the kilovolt range) and high frequencies of typically about 200 kHz to 4 MHz strong electromagnetic fields are created during activation (lower frequencies in the ultrasound range between 20 kHz to 200 kHz can be used, too, depending on the type of electrosurgical instrument, e.g. an ultrasound instrument), thereby allowing the energy harvesting circuit to draw energy from these electromagnetic fields and to provide it as operating power to the electronic circuits of the electrosurgical instrument, in particular its signal processing device. This allows for a proper supply of a rather powerful signal processing device, thereby enabling it to accomplish even demanding tasks in terms of acquiring and processing a larger amount of and dynamically changing data, which may be real-time data.

The invention draws on the finding that providing substantial energy for components of the electrosurgical instrument itself is key to supply more powerful electronic and electric components at the instrument, thereby enabling it for enhanced data acquisition and processing. Gathering, processing and transmitting of dynamic and live, preferably real-time, data to and from the electrosurgical generator is thus made possible. Furthermore, sensors and/or even actuators on the electrosurgical instrument could be properly powered, thereby providing additional functionality.

Sensors could be of any type. Typically, the sensor may be e.g. a temperature sensor configured to detect temperature of an instrument tip, in particular a HF applicator, and/or a sensor detecting an open or closed state of movable jaws, a sensor configured to detect movement of the electrosurgical instrument and/or its tip, a sensor for detecting status of the tissue to be treated or whether contact to the tissue is made, a sensor sensing status of components and/or materials of the electrosurgical instruments itself, like usage data and/or wear data.

The actuator may be an actual electric/electromechanical actuator in a strict sense, like actuating jaws of a pincer of the electrosurgical instrument, and/or in a broader sense devices which do something like presentation of signals by means of a display placed at the electrosurgical instrument or other signalling elements, e.g colored lighting indicative for various conditions and states of the electrosurgical instrument and/or the electrosurgical generator to which said instrument is connected.

The term HF means high frequency and encompasses frequencies in the radio frequency range (typically 200 kHz to 4,000 kHz) and but may also encompass frequencies in the ultrasound frequency range (typically 20 kHz to 200 kHz).

By the term “activation” and by the terms “activated” or “non-activated” it is meant that the electrosurgical instrument is actually energized with HF energy (to be supplied by the electrosurgical generator).

It is to be noted that the wireless communication interface and the energy harvesting circuit are separate entities. The wireless communication interface may be in particular a RFID transponder which can be actively powered or passively.

Advantageously, the energy harvesting circuit is mounted at the HF energy cable or at an internal HF energy conductor of the electrosurgical instrument. Mounting at the HF energy cable, preferably at the plug thereof, allows for a positioning of the energy harvesting circuit closer to the electrosurgical generator, thereby minimizing the distance for the wireless communication. Further, the body of the electrosurgical instrument can be made smaller since no energy harvesting circuit needs to be placed inside. Conversely, by placing the energy harvesting circuit within the body of electrosurgical instrument itself the HF energy cable can be kept flexible, pliable and light which can be beneficial for making the electrosurgical instrument more manoeuvrable.

The energy harvesting device is preferably provided with an energy storage device. Thereby, energy gained by the energy harvesting device can be stored, and can subsequently be supplied to the signal processing device for subsequent periods when no further energy can be harvested, e.g. due to lack of activation of the electrosurgical instrument. Thereby, a continuous operation of the electronics, in particular the signal processing device, of the electrosurgical instrument can be maintained even if the electrosurgical instrument is only activated from time to time. This allows for a more stable operation of the electronic circuits, in particular the signal processing device. The energy storage device is advantageously embodied as a super capacitor. This enables storage of a sufficient amount of energy and has the advantage of allowing high charging currents, thereby requiring only a minimum of time for filling up of the storage. However, a super capacitor is not a must, the energy storage device can also be a conventional accumulator, like a lithium ion cell or similar. It is further beneficial that the energy storage is enabled to power the electrosurgical instrument even during longer periods of time of non-activation, including the electrosurgical instrument being disconnected from the electrosurgical generator. This allows for periodic monitoring of essential parameters, e.g. environmental parameters like temperature and humidity in order to check for acceptable storing conditions. By such monitoring operational safety of the electrosurgical instrument can be enhanced.

Preferably, a microcontroller is provided for the signal processing device. Such a microcontroller is enabled to perform a plurality of different functions, up to but not limited gathering of various sensor data and even controlling an actuator, if provided, at the electrosurgical instrument. In particular, a combination of said microcontroller with the energy storage is advantageous since by virtue of the energy storage a reliable constant operation of the microcontroller can be achieved without any interruption due to lack of energy.

Further, owing to said energy storage the microcontroller can be enabled to operate even if the electrosurgical instrument non-activated for a prolonged period of time or even if it is disconnected from the generator. This allows the microcontroller to perform additional tasks, like being configured to monitoring environmental conditions, e.g. temperature and/or humidity, by means of suitable sensors at the electrosurgical instrument. Preferably, the microprocessor is configured for a sleep and/or hibernate state and further preferably a wake-up device is provided configured to waking up the microprocessor on predetermined conditions, e.g. a certain time interval, only thereby saving energy and allowing long-time monitoring. The microprocessor is further configured to signal any exceedance prior to the next use, e.g. if overtemperature occurred during storage. This ensures operational safety and lessens risks due to otherwise undetected deterioration of the electrosurgical instrument.

In an advantageous embodiment, a communication control device is provided, wherein said communication control device is configured to block communication via the wireless communication mean when the electrosurgical instrument is in an activated state. Thereby communication can be interrupted during activation of the electrosurgical instrument.

Interference on the wireless communication induced by the high-frequency high-voltage as produced by the electrosurgical generator can be avoided, thereby keeping the quality of the wireless communication high. It was realized that in many instances it is not necessary to have constant data communication, rather it is preferable to allow some brief pauses, namely during actual activation of the electrosurgical instrument. Thereby, quality of the electronic data transmission via the wireless communication interface can be maintained at a high level even for dynamic data, without any appreciable loss of information density or speed.

For detecting of the activated state of the electrosurgical instrument, a handswitch may be employed. This has the advantage that such a handswitch is often provided at the electrosurgical anyway in order to trigger activation of the electrosurgical generator, and by tapping the output signal of the handswitch an effective blocking of communications via the communication control device can be achieved without requiring additional hardware. However, not all instruments are activated by a handswitch on the electrosurgical instrument proper. Especially in those cases it is preferable to have provided an automatic detecting circuit that is configured to determine when the electrosurgical instrument is activated, preferably by assessing the magnitude of HF energy supplied to the instrument. If the supplied HF energy is beyond a threshold value, then activation of the instrument can be assumed. It is to be noted that the actual threshold value may be different from instrument to instrument, and that the threshold value may be stored within the memory of the electrosurgical instrument proper. Thereby, a precise automatic determination of activated status can be achieved, with regard to the special characteristics of each instrument.

Further, preferably a data transfer device is provided which is operatively connected to the wireless communication means and to the signal processing device. The data transfer device allows for a controlled and safe data transfer of the electrosurgical instrument to and from the electrosurgical generator supplying the energy. In a particularly preferred embodiment, the data transfer device also acts as the memory, preferably it is embodied as a dual port memory. This allows reading or writing of data, in particular measurement data, control inputs/outputs and/or settings of the electrosurgical instrument to or from one side (port) of the dual port memory. Via the wireless communication means, the electrosurgical generator can read and write to the other side (port) of this dual port memory, in particular during phases when the electrosurgical instrument is not activated. Further preferably, the data transfer device is powered by the communication and data means, e.g. by employing a RFID transponder. Thereby energizing of this device, in particular the dual port memory, is ensured at all times and not limited to the energy provided by the energy harvesting circuit and its storage. This is an important safety feature as thereby a basic communication functionality becomes independent from actual activation of the electrosurgical instrument, i.e. such basic communication is guaranteed.

In an advantageous embodiment, a matching circuit is provided which is configured to match an antenna of the energy harvesting circuit to a frequency of the HF energy supplied. By virtue of such matching, power transfer is improved to the energy harvesting circuit for supplying the signal processing device and other circuits of the electrosurgical instrument. Preferably, the matching circuit may be configured such as to provide an impedance matching. As this matching is dependent on the actual frequency or frequencies used for HF energy, the matching circuit is preferably switchable for an adaption to a second, different frequency of the HF energy. To this end, it encompasses additional circuitry, like additional inductance, special filters, and/or additional capacitors that are preferably switchable for adaption to said second different frequency. Thereby, an optimized power transfer to an efficient operation of the energy harvesting device can be reached even if different frequencies are to be employed for HF energy. This can be of particular importance if different modes are selectable for operation of the electrosurgical instrument, as dependent on the circumstances these different modes may comprise usage of different HF frequencies. Thereby, by appropriate switching of the matching circuit a continuously optimized power transfer can be ensured.

Preferably, the electrosurgical instrument is configured such as to operate in a first state wherein energy is harvested and the signal processing device, in particular a microcontroller thereof, and/or the at least one sensor and/or actuator is run, and/or the memory is accessed by the signal processing device; and a second state wherein the wireless communication means is activated for data transmission to/from the electrosurgical generator, wherein the first state is selected during activation of the electrosurgical instrument and the second state is selected when the electrosurgical instrument is not activated. By virtue of these states, an energy harvesting can be performed efficiently in the first state, whereas a safe and reliable and efficient data transfer can be accomplished in the other, second state. Thereby, adverse influence of powerful electromagnetic fields that may be present during activation on the wireless communication can be minimized. This is important since many low-power wireless communication technologies, as RFID transponders or Bluetooth, due to their very low power nature are rather sensitive to electromagnetic interference and disturbances by powerful electromagnetic fields as they are prevalent in the context of an electrosurgical instrument having activated its HF supply. By providing several states for “energy harvesting” at one hand and “data transfer” at the other hand. a well determined and robust system design can be achieved which provides for safe, efficient and reliable data communication to and from the electrosurgical instrument. As a result, any risks that may be created by communication errors due to interference are thereby avoided, leading as a result to a reduced risk for the patient. Actual selection of the first or second state, resp., is preferably driven by the electrosurgical instrument, not by the electrosurgical generator.

The invention further relates to an assembly of an electrosurgical instrument as described above and an electrosurgical generator, the electrosurgical generator comprising a control unit controlling an inverter for generating a HF energy being supplied to an output socket configured for plugging of a connector to the electrosurgical instrument, thereby supplying the electrosurgical instrument. With respect to the interface data communication with the electrosurgical generator reference is made to the above description.

Preferably, the electrosurgical generator comprises a transceiver for close range communication, said transceiver being connected to the main control unit and being operatively connected to a complementary transponder of the electrosurgical instrument. Close range communication in the context of the present invention is generally confined to the same room (operating room), and can be in particular low-power wireless communication, e.g. like the technology used for RFID transponder, Bluetooth, near field communication (NFC) etc.

The invention is explained in more detail below with reference to an advantageous exemplary embodiment. In the figures:

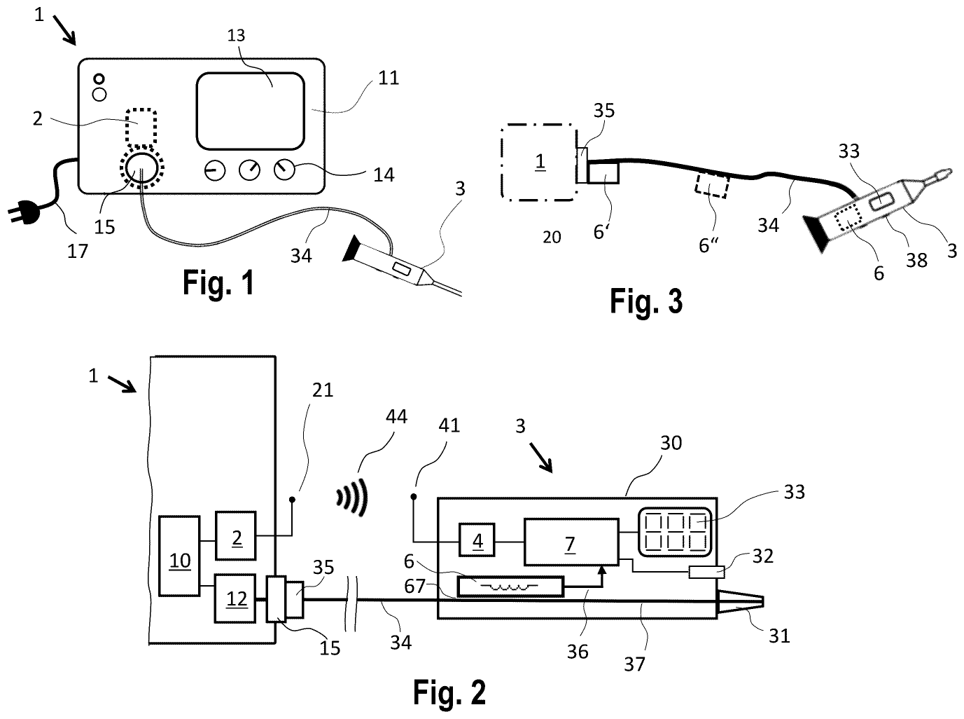

FIG. 1 shows a frontal view of an electrosurgical generator with an attached electrosurgical instrument according to a first embodiment of the invention;

FIG. 2 shows a block diagram of the electrosurgical instrument of the first embodiment and its attachment to the electrosurgical generator;

FIG. 3 shows variants of the electrosurgical instrument according to a second and third embodiment;

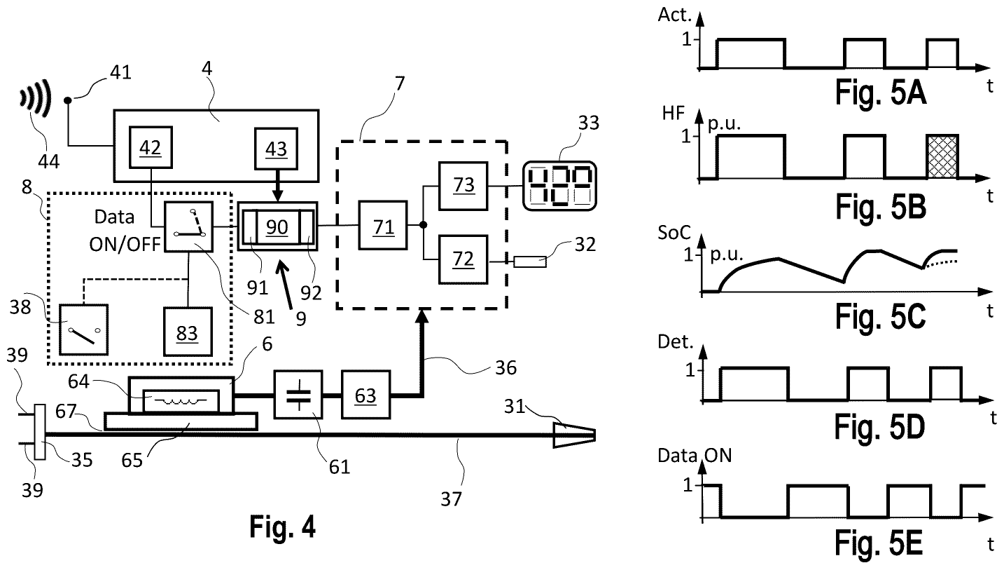

FIG. 4 shows a schematical functional diagram of various functional entities of the electrosurgical instrument of the first embodiment; and

FIG. 5A-E show various graphs concerning activation status, HF energy supply, state of charge (SoC), status of a detector, and status of data transfer.

An exemplary embodiment for an assembly of an electrosurgical instrument according to a first embodiment of the present invention and an electrosurgical generator supplying energy to the electrosurgical instrument is illustrated in FIG. 1. The electrosurgical generator which is designated as a whole by reference numeral 1 comprises a housing 11 having an output socket 15 for connection of the electrosurgical instrument 3. Said electrosurgical instruments 3 is connected via an HF energy cable 34 with a plug 35 having two prongs 39 at its end to be inserted into the socket 15. For communication of the electrosurgical generator 1 with the electrosurgical instruments 3, a wireless communication frontend 2 is provided which is configured for wireless communication with the electrosurgical instrument 3 attached to socket 15, as will be described later in more detail. The electrosurgical generator 1 is further provided with a user interface comprising a display 13 which may be a touchscreen and/or knobs 14 may be present for inputs by a user.

A power supply cable 17 is provided for connection to an electrical power source (not shown) which may be an electricity grid, like AC mains in a building, or an off-grid source of electric energy, like a 12 Volt or 24 Volt battery of a vehicle or in a mobile hospital. This electrical energy is fed to an inverter 12 (see FIG. 2) which is configured to generate HF energy which is output from the electrosurgical generator 1 by its output socket 15, to which the electrosurgical instruments 3 is to be connected by means of its plug 35. The inverter 12 generates alternating current in a selectable voltage range up to a few kilovolts as selectable frequencies up to 4000 kHz as it may be as low as 200 kHz, or in specific cases like an electrosurgical generator 1 being enabled for driving an ultrasound instrument the generated frequency may also be in the ultrasound range from about 20 kHz to 200 kHz. The electrosurgical instrument 3 which is to be plugged into the output socket 15 can accordingly be supplied with radiofrequency energy as well as ultrasound energy, or a combination thereof, by means of the HF energy cable 34.

Constitution and operation of the electrosurgical generator 1 is conventional and is known to the person skilled in the art, therefore it will not be addressed here in more detail for the sake of brevity. It may suffice to state that operation of the electrosurgical generator 1 as a whole and specifically its inverter 12 is controlled by a main control unit 10 which is internally connected to various internal components (not shown) including said inverter 12 and a wireless communication frontend 2 which is, by means of its antenna 21, configured for wireless communication via a radio link 44 with the electrosurgical instrument 3 and its corresponding instrument antenna 41. The preferred wireless communication may be e.g. RFID technology, wherein the electrosurgical instrument 3 with its wireless communication means acts as a RFID transponder. However, other suitable wireless technologies may be employed also, e.g. but not limited to Bluetooth, Wireless LAN or Nearfield Communication (NFC).

The electrosurgical instrument 3 comprises a main body 30 having an internal HF energy conductor 37 for conveying the HF energy supplied by the cable 34 to an HF applicator 31 which is configured for transferring said HF energy to tissue to be treated (not shown), and a handswitch 38 for activating the supply of HF energy (alternatively, a—not shown—footswitch could be used, too). The HF applicator 31 may e.g. be unipolar, like a blade, or bipolar, like a pincer, but may have other constitutions. The electrosurgical instrument 3 further comprises a signal processing device 7 and a wireless communication means 4 to which the instrument antenna 41 is connected, thereby being enabled for gathering and communicating real-time data. Further, the electrosurgical instrument 3 may be provided with at least one sensor 32 and/or at least one actuator 33.

The sensor 32 may for example be a temperature sensor configured to detect temperature of the instrument tip, in particular of the HF applicator 31; a sensor detecting an open or closed state of movable jaws, in particular if the HF applicator 31 is configured as a pincer; a sensor configured to detect movement of the electrosurgical instrument 3 and/or its tip, e.g. the HF applicator 31; a sensor for detecting contact status of the tissue to be treated; and/or a sensor sensing status of components and/or materials of the electrosurgical instruments 3 itself, like (internal) temperature, usage data and/or wear data.

The actuator 33 may be an actual electric/electromechanical actuator and/or a display placed at the electrosurgical instrument 3 itself. This allows convenient, prompt and direct presentation of information in the primary field of view of the surgeon. This can be in particular parameters determined by the sensor 32 of the electrosurgical instruments 3 itself, other parameters which are conveyed via the radio link 44 from the wireless communication frontend 2 of the electrosurgical generator 1, or important status information about HF energy delivery, e.g. information about the next pulse of HF energy in a complex progressive mode of the electrosurgical instrument 1. Thereby, the surgeon is kept informed about the next step of HF energy delivery without needing to turn his head toward the main display 13 of the electrosurgical generator 1, rather can stay focused with his eyes on the site of operation and the electrosurgical instruments 3 itself.

Moreover, the actuator 33 can be a signaling element like a lighting, e.g. of buttons and/or the handswitch 38 of the electrosurgical instruments 3. Particularly, it can be used for colour coded lighting thereby providing additional information to the surgeon, like providing a yellow light at the handswitch 38 indicative for “stopping activation” (flashing red light: “immediately stopping”) due to reaching a critical high (flashing red light: excessive) temperature sensed at the applicator tip 31 or surrounding tissue as sensed by other sensors 32 of the electrosurgical instrument 3.

The at least one sensor 32 and/or the at least one actuator 33 are connected to the signal processing device 7. The signal processing device 7 is configured to process the data, and in particular is optionally enabled to process it in real-time. Parameters which are gained by processing and gathering data are communicated to the wireless communication means 4 which is configured to transmit this data by the instrument antenna 41 and the radio link 44 to the antenna 21 and the wireless communication frontend 2 and ultimately to the main control unit 10 of the electrosurgical generator 1. Thereby, using actually gathered data of the electrosurgical instrument 3, controlling the electrosurgical generator 1 and its operation as well as supplying the electrosurgical instrument 3 with HF energy in accordance with actual and dynamic, preferably real-time, data gathered at the electrosurgical instrument 3 is enabled. Important and highly variable parameters, like temperature of the applicator tip 31 of the electrosurgical instrument 3 and/or of surrounding tissue can therefore be provided to the control unit 10 for a much improved dynamic control of supply of HF energy and therefore improved performance of the electrosurgical instrument 3. This even allows more sophisticated modes of operation of the electrosurgical generator 1 since such modes of operation are, owing to the invention, enabled to use and consider actual and dynamic data from the electrosurgical instruments 3 and tissue to be treated.

Conversely, actual and time critical data may be provided in the opposite direction by the electrosurgical generator 1, in particular its main control unit 10, via the wireless communication frontend and its antenna 21 to the electrosurgical instruments 3 by other radio link 44 and the instrument antenna 41 to the wireless communication means 4 of the electrosurgical instrument 3. Thereby important signalling, e.g. like reaching of specific target temperature or an alert to the surgeon, can be provided directly to a electrosurgical instrument 3. Further, an actuator 33 of the electrosurgical instrument 3, e.g. configured for opening or closing jaws of a pincer, can be directly performed, thereby facilitating fast and effective control by the main control unit 10 of the electrosurgical generator 1.

Such improved operation requires high performance of the signal processing device 7 as well as of the digital communication means 4, in addition to satisfying the power demands of the at least one sensor 32 and/or the at least one actuator 33. To this end, an energy harvesting circuit 6 is provided for wirelessly harvesting energy from the HF energy supplied by the cable 34 to the electrosurgical instrument 3. The energy harvesting circuit 6 is located in close proximity but not in direct contact to the energy cable 34 or to the internal energy conductor 37. To avoid direct contact and therefore galvanic contacting, an isolation gap 67 is provided between the energy harvesting circuit 6 and the HF energy cable 34/energy conductor 37. The gap may be free air, but preferably is filled with isolating material.

The energy circuit 6 is depicted in FIG. 3 as being an internal component of the electrosurgical instrument 3, according to a first embodiment of the invention. A second embodiment features the energy harvesting circuit 6′ (shown by a solid line in FIG. 3) to be positioned at the instrument plug 35 at the very beginning of the HF energy cable 34, and a third embodiment features the energy harvesting circuit 6″ to be positioned somewhere along the HF energy cable 34, e.g. at a middle portion thereof (as shown by dashed lines in FIG. 3).

The energy harvesting circuit 6 is provided with an energy harvesting antenna 64 which is configured to pick up energy from electromagnetic fields as formed by the HF energy being conveyed through the cable 34/conductor 37. The harvesting antenna 64 may be embodied as an inductive pickup, e.g. a coil.

Optionally, a matching circuit 65 may be provided for the energy harvesting circuit 6. The matching circuit 65 is configured to match an antenna 64 of the energy harvesting circuit to a frequency of the HF energy supplied via the cable 34 and/or the energy conductor 37. Depending on the type of electrosurgical instrument 3, it may be possible to supply the HF energy while using different frequencies, e.g. in the radiofrequency range or ultrasound frequency range. In either case it is desirable to be able to energise the energy harvesting circuit 6 regardless whether the HF energy supplied is in the radiofrequency range or ultrasound frequency range. As antennas like the harvesting antenna 64 are usually designed to a specific frequency or frequency range (e.g. radiofrequency range), it will be necessary to provide additional measures in order to make this harvesting antenna 64 effective for a different frequency range (e.g. ultrasound frequency range). This is realized by the matching circuit 65 which encompasses additional circuitry, like additional inductance, special filters, and/or additional capacitors that are preferably switchable for adaption to the other different frequency or frequency range. Thereby, an improved matching of the energy harvesting antenna 64 to the different frequency employed for supplying the HF energy can be achieved by the matching circuit 65.

The energy thereby gathered by the harvesting antenna 64 is supplied to an energy storage 61 which may be embodied as an accumulator, e.g. of the lithium ion type, or preferably as a super capacitor. The latter option is preferable due to lighter weight and being capable of faster charging/discharging.

Further, a voltage regulator 63 is provided which is supplied by the energy storage 61 and delivers regulated voltage via an internal operational power wire 36 to the signal processing device 7 and, if required, to the wireless communication means 4. Thereby, in particular the signal processing device 7 as well as the at least one sensor 32, in particular a plurality of sensors or complex sophisticated sensors requiring additional energy, as well as the at least one actuator 33 are reliably provided with sufficient energy for operation. Since rather strong electromagnetic fields are formed by the HF energy conveyed through the cable 34/energy conductors 37, a considerable amount of energy can be harvested by the energy harvesting circuit 6, thereby enabling proper supply of even powerful components, like a microcontroller 71 and actuators 33 at the electrosurgical instrument 3. Moreover, not only state-of-the-art microcontrollers 71 can be powered, but also proper input/output devices like high-performance analog/digital-converter (ADC) 72 and or digital/analogue-converters (DAC) 73 can be powered owing to the energy harvesting circuit 6.

In order to transfer measured data as acquired by the at least one sensor 32 or computed by the microcontroller 71 and to transfer other input and output data via the radio link 44, a data transfer device 9 is provided between the signal processing device 7 and the wireless communication means 4. The data transfer device 9 comprises a memory for data to be transferred, said data transfer device 9 preferably comprising a dual port memory 90. Such a dual port memory 90 allows for an asynchronous data collection and gathering at one hand and wireless transfer at the other hand. Thereby, a transferring of data into the dual port memory 90 is enabled from a first side 91, however it is generally advisable to transfer such data via the radio link 44 only during such periods when the electrosurgical instrument 3 is not energised in order to reduce adverse impact of interference by the electromagnetic field of the HF energy conveyed to the electrosurgical instrument 3. Storing and retrieving the data by the signal processing device 7 and conversely writing of data and signals can be performed via the second side port 92 at all times, irrespective whether wireless transfer via the first side port 91 is actually performed or not.

Energy for operation of the data transfer device 9, particularly its dual port memory 90, can be provided by the energy harvesting circuit 6 likewise as energy is provided for the signal processing device 7. However, depending on the kind of radio link 44 used it may also be feasible to provide energy by the wireless communication means 4. For example, in the case of RFID technology employed for the radio link 44, RFID transponders exist which are capable of being self-powered by the energy of the radio waves used. If such RFID technology is to be employed, then the wireless communication means 4 may comprise a data branch 42 conveying the data, as described above, and an energy branch 43 providing a, quite tiny, amount of energy for energising an ultra-low-power device like said dual port memory 90. Thereby, an alternative energy supply for the dual port memory 90 can be realised, or an additional energy supply can be achieved thereby essentially resulting in a dual energy supply with the benefit of increased fail safety. Providing energy to the dual port memory 90 by means of the energy branch 43 of the wireless communication means form a significant advantage in that proper energy supply to the dual port memory 90 is ensured even for long periods of non-activation of the electrosurgical instrument 3. In consideration of the low power available via the energy branch 43 it is highly advisable to restrict this kind energising to the dual port memory 90 only (and not any of the other components of the electrosurgical instrument 3).

As already indicated above, it may be desirable to constrain actual data transmission via the radio link 44 only to those times wherein the electrosurgical instrument 3 is not activated. This allows minimising of any risk of an adverse impact to the wireless data transmission stemming from interference due to the strong electromagnetic fields induced by generating the HF energy electrosurgical generator 1 and conveying said HF energy to the electrosurgical instrument 3. To this end, a communication control device 8 is provided which comprises a blocking unit 81 configured to block the data transmission via the wireless communication means 4, in particular its data branch 42. The communication control device 8 is configured to be active upon activation of the electrosurgical instrument 3, i.e. when HF energy is provided to said electrosurgical instrument 3. Detection of such activation can be performed either by tapping off a signal of the handswitch 38 used for activating the electrosurgical instrument 3, or by providing an automatic detection circuit 83 that is enabled to automatically detect when the electrosurgical instrument 3 is activated. This could be accomplished by providing a sensor for the electromagnetic fields which are present upon application of HF energy to the electrosurgical instrument 3. Such an automatic detection has the advantage that it also works reliably if activation is commanded by other means, e.g. by a foot switch. The automatic detection circuit 83 may also be communicatively connected to the energy harvesting circuit 6, thereby detecting activation of the electrosurgical instrument 3 whenever the energy harvesting circuit 6 is actually harvesting energy from the HF energy supplied to the electrosurgical instrument 3 in this moment. Thereby the need for any additional sensor can be avoided. Either way, if activation of the electrosurgical instrument 3 is detected by either the handswitch 38 or the automatic detection circuit 83, then the blocking unit 81 will be activated which effectively shuts down data communication via the radio link 44, thereby effectively isolating communication of the first side port 91 of the dual port memory 90. By virtue of this, any adverse effect of the strong electromagnetic fields accompanied by the delivery of HF energy to the electrosurgical instrument 3 can be reliably avoided, thereby enhancing error immunity of the wireless communication 44 thus improving operational safety due to reduced risk of errors.

Operation and timing are visualized by various graphs in FIG. 5A to E. FIG. 5A shows activation status of the electrosurgical instrument 3, that is the signal shown is high (“1”) whenever the electrosurgical instrument 3 is to be activated. Accordingly, shown in the graphs are three instances when the electrosurgical instrument 3 is to be activated, namely a first longer instance, a second instance and a brief third instance. Owing to such activation, the electrosurgical generator 1 provides HF energy to the electrosurgical instrument 3 as shown in FIG. 5B for each of the instances. In the present example, during the third instance the electrosurgical generator 1 delivers HF energy at a different frequency than that for the first two instances, e.g. in the ultrasound frequency range as opposed to the radio frequency range employed for the first two instances. When such HF energy is supplied to the electrosurgical instrument 3, then the HF energy harvesting circuit 6 is harvesting energy from the cable 34 or the energy conductors 37 and supplies it to the energy storage 61, thereby increasing the state of charge (SoC) of said storage as shown in FIG. 5C. As can be appreciated, during activation (see FIG. 5A) and therefore supplying of HF energy (see FIG. 5B) to the electrosurgical instrument 3, energy is captured by the energy harvesting circuit 6 and the state of charge of the energy storage increases during the activation period. Between the activation periods, the signal processing means 7, in particular the microprocessor 71 in conjunction with the at least one sensor 32 and/or the at least one actuator 33, continuously draw energy and therefore the state of charge (SoC) gradually diminishes. The state of charge (SoC) will increase again during the next activation instance. For the third instance using a different frequency, the harvesting antenna 64 of the energy harvesting circuit 6 is mismatched due to the different frequency, which would inevitably result in a lesser energy capture as is indicated by the dotted line in FIG. 5C. However, due to the matching circuit 65 an adaption to said different frequency is achieved and therefore an improved energy harvesting can be accomplished, thereby resulting in a higher raise of the state of charge (SoC) during the third instance of activation. This is shown by the solid line being higher than the dotted line.

The output of the automatic detection circuit 83 is shown in FIG. 5D. As it can be seen, activation of the electrosurgical instrument 3 is reliably detected. Accordingly, whenever the activation is detected, the blocking unit 81 will be activated and therefore data flow between the dual port memory 90 and the wireless communication means 4 will be blocked, whereas in the meantime between activations the data flow will be switched on, as shown in FIG. 5E. Thereby any interference between the HF energy and the data transfer via the radio link 44 is effectively avoided.

1. An electrosurgical instrument having a HF energy cable configured for being supplied with HF energy from an electrosurgical generator, the electrosurgical instrument comprising a memory comprising status information of the said electrosurgical instrument and wireless communication means configured for communicating data signals between said memory and a corresponding wireless communication frontend of the electrosurgical generator,
wherein
the electrosurgical instrument further comprises a signal processing device configured for gathering and communicating gathered data of said electrosurgical instrument, the signal processing device being communicatively connected to the wireless communication means and comprising
at least one sensor and/or actuator at the electrosurgical instrument, and an energy harvesting circuit configured for wirelessly harvesting energy from the HF energy supplied by the cable, said energy harvesting circuit being a power supply for the signal processing device and/or the at least one sensor and/or the at least one actuator.

2. The electrosurgical instrument of claim 1, wherein the energy harvesting circuit is mounted at the HF energy cable or at an internal HF energy conductor of the electrosurgical instrument.

3. The electrosurgical instrument according to claim 1, wherein the energy harvesting circuit is provided with an energy storage device.

4. The electrosurgical instrument according to claim 1, wherein a microcontroller is provided for the signal processing device.

5. The electrosurgical instrument according to claim 1, wherein a communication control device is provided, said communication control device being configured to block communication via the wireless communication interface when the electrosurgical instrument is in an activated state.

6. The electrosurgical instrument according to claim 5, wherein the activated state is determined by activation of an handswitch of the electrosurgical instrument and/or by an automatic detecting circuit, depending on a magnitude of HF energy supplied.

7. The electrosurgical instrument according to claim 1, wherein a data transfer device is provided which is operatively connected to the wireless communications means and to the signal processing device.

8. The electrosurgical instrument according to claim 7, wherein the data transfer device acts as the memory.

9. The electrosurgical instrument according to claim 8, wherein the data transfer device is powered by the wireless communication means.

10. The electrosurgical instrument according to claim 1, wherein a matching circuit is provided which is configured to match an antenna of the energy harvesting circuit to a frequency of the HF energy supplied.

11. The electrosurgical instrument according to claim 10, wherein the matching circuit is switchable for an adaption to a second, different frequency of the HF energy.

12. The electrosurgical instrument according to claim 11, wherein a switching of the matching circuit depends on a mode selected for operation of the electrosurgical instrument.

13. The electrosurgical instrument according to claim 1, wherein the electrosurgical instrument is configured so as to be operated
in a first state wherein energy is harvested and the signal processing device and/or the at least one sensor and/or the at least one actuator is run and/or the memory is accessed by the signal processing device; and
a second state wherein the wireless communication means is activated for data transmission to/from the electrosurgical generator,
said first state being selected during activation of the electrosurgical instrument and said second state being selected when the electrosurgical instrument is not activated.

14. An assembly of an electrosurgical instrument according to claim 1 and an electrosurgical generator comprising a main control unit controlling an inverter for generating a HF energy being supplied to an output socket configured for plugging of a connector to the electrosurgical instrument.

15. The assembly of claim 14, wherein the electrosurgical generator comprises a transceiver for close range communication being connected to the main control unit and being operatively connected to a complementary transponder of the electrosurgical instrument.

16. The electrosurgical instrument according to claim 7, wherein the data transfer device is embodied as a dual port memory.
